(19) United States
(12) Patent Application Publication
(10) Pub. No.: US 2013/0005049 A1
(43) Pub. Date: Jan. 3, 2013

(54) DEVICES AND METHODS FOR DETECTING SINGLE NUCLEOTIDE POLYMORPHISMS

(75) Inventors: Hanbin Mao, Kent, OH (US); Deepak P. Koirala, Kent, OH (US)

(73) Assignee: KENT STATE UNIVERSITY, Kent, OH (US)

(21) Appl. No.: 13/485,201

(22) Filed: May 31, 2012

Related U.S. Application Data
(60) Provisional application No. 61/571,558, filed on Jun. 30, 2011.

(51) Int. Cl. G01N 33/53

(52) U.S. Cl. 436/501; 422/69

(57) ABSTRACT

A device for detecting Single Nucleotide Polymorphism (SNP) and associated methods has been described. The stochastic behavior of a single-molecule probe is utilized to recognize wild type and SNP sequences in a microfluidic platform using a laser-tweezers instrument. The mechanical signal provides substantially noise free sensing with high sensitivity and the selectivity. The method has an inherent capacity to develop as a generic biosensor using other recognition elements such as aptamer for example.

### FIELD OF THE INVENTION

The present invention relates to a device for detecting single nucleotide polymorphism (SNP) and associated methods. The stochastic behavior of a single-molecule probe is utilized to recognize wild type and SNP sequences in a microfluidic platform using a laser-tweezers instrument. A mechanical signal provides substantially noise free sensing with high sensitivity and the selectivity. The method has an inherent capacity to develop as a generic biosensor using other recognition elements such as aptamer for example.

### BACKGROUND OF THE INVENTION

SNP is a common genetic variation in human genome with an average occurrence of ˜1/1000 base pairs. SNP detection is crucial for biological and clinical aspects since it is associated with diseases, anthropometric characteristics, phenotypic variations and gene functions. Recent strides towards personalized medicines necessitate high resolution genetic markers to track disease genes, which further amplifies the importance of SNP detection.

Most SNP detecting methods use amplification steps such as PCR to achieve highly sensitive detection. However, efficiency of PCR is dependent on the target sequence. Recently, Mirkin and co-workers, see Taton, T. A.; Mirkin, C. A.: Letsinger, R. L. Science 2000, 289, 1757-1760; and Nam, J.-M.; Stoeva, S. I.: Mirkin, C. A. J. Am. Chem. Soc. 2004, 126, 5932-5933 developed alternative nano-particles based amplifications and attained femto molar detection limits. Methods incorporating amplification steps require, laborious and time consuming multi-step protocols, which may expose a sample to uncontrollable human and environmental factors. Approaches that employ less amplification steps, such as molecular beacon, see Tyagi, S.: Kramer, F. R. Nat Biotech 1996, 14, 303-308; and Tan, W.; Wang, K.; Drake, T. J. Curr. Opin. Chem. Biol. 2004, 8, 547-553, can reportedly reduce these disadvantages. Yet, fluorescence based detection often suffers from indigenous background that deteriorates detection limit.

Various attempts to combine laser tweezers with a lab on a chip system are known, for example Gross, P. et al. in methods in Enzymology; Academic press: 2010; Vol. 475, p 427-453; and Enger, J. et al. Lab on a Chip 2004, 4, 196-200. However there is still a need for a device and method that utilize this system to demonstrate bio-sensing at a single molecule level.

In view of the above, a problem of the invention is discovering how to avoid or reduce sophisticated amplification steps while at the same time providing desirable detection limits and selectivity in reasonable detection time. The method disclosed herein presents a first example of the force based stochastic sensing of SNP at a single molecule level.

### SUMMARY OF THE INVENTION

In view of the above, it is an object of the present invention to provide devices and methods which utilize a force based sensing of SNP at a single-molecule level. The single; molecule nature of the SNP-probe allows for stochastic sensing that presents high sensitivity and selectivity.

Yet another object of the invention is to provide a device that utilizes a mechanical signal to sense SNP that is subject to little environmental interference while providing high signal to noise ratio.

Still another object is to provide a device and method that utilizes two stages, for example on-off, mechanical signals of a single DNA template that recognizes SNP that are recorded by a laser tweezers device in a microfluidic platform.

A further object is to provide a device including a SNP-probe comprising a hairpin that recognizes a SNP sequence, with the probe selectively placed inside a microfluidic device, wherein the laser tweezers is utilized to provide force based SNP sensing.

An additional object of the invention is to provide a method for sensing with a laser tweezers a wild type DNA sequence or a SNP sequence by allowing binding of the same with the SNP-probe in a microfluidic platform. In a further step wild type sequence or SNP sequence is determined by measuring the force required to eject the bound target during the extension of the target bound SNP-probe.

Accordingly, in one aspect of the present invention, a device for detecting a single nucleotide polymorphism (SNP) is disclosed comprising a SNP-probe including a hairpin that recognizes a target DNA comprising one or more of a wild type and SNP sequences; a microfluidic device; and a laser tweezers device operatively connected to the microfluidic device for force based stochastic sensing of the one or more of the wild type and the SNP sequences.

In another aspect of the present invention, a method for detecting a single nucleotide polymorphism (SNP) is disclosed comprising the steps of obtaining a SNP detection device including a microfluidic device operatively connected to a laser tweezers device; connecting a SNP-probe containing a hairpin that recognizes a SNP sequence to the SNP detection device; and measuring a force exerted by the SNP-probe in the SNP detection device in the presence of a target sample and determining whether the SNP sequence is present in the target sample.

### BRIEF DESCRIPTION OF DRAWINGS

The invention will be better understood and other features and advantages will become apparent by reading the detailed description of the invention, taken together with the drawings, wherein:

FIG. 1 schematically illustrates a SNP-probe containing a hairpin that recognizes a SNP sequence, wherein the hairpin is sandwiched between two handles, here dsDNA handles, which are tethered to two optically trapped beads via digoxigenin (Dig)-antiDig antibody and biotin-streptavidin linkages;

FIG. 2 schematically illustrates a sensing mechanism for the DNA targets, wherein a buffer channel hosts the SNP-probe that hops between folded and unfolded hairpin states, wherein the SNP-probe is then moved via the conduit to the target channel in which a sample DNA target (Wild type or SNP) is present, wherein a specific recognition between the SNP-probe and the target terminates the hopping of the hairpin;

FIGS. 3A-D illustrate detection of a specific DNA target, CMP1 by stochastic hairpin hopping, wherein FIG. 3A illustrates a force vs. extension curve during mechanical stretching and relaxing of the SNP-probe in the buffer channel, FIG. 3B shows force vs. time traces observed for SNP-probe at fixed optical trap positions in the buffer (top) and the target (bottom) channel, FIG. 3C is a force vs. extension curve of the SNP-probe in the target channel where folding-unfolding features were not observed, and FIG. 3D illustrates hopping traces for a SNP-probe with different CMP1 concentrations wherein the vertical dotted line indicates the transfer of the SNP-probe from the buffer to the target channel, the two headed arrows depict the time observed before the hopping ceases to the unfolded hairpin state, which indicates the binding of the CMP1 to the hairpin;

FIGS. 4A-C illustrate differentiation between a wild type and SNP sequence, wherein FIG. 4A illustrates typical force vs. extension curves for CMP1 or MUT1 bound to a SNP-probe wherein the darker and lighter arrows represent the stretching and relaxing curves, respectively, the left inset shows a feature due to the ejection of the bound target, the right inset shows the refolding of the hairpin, which indicates the regeneration of the probe, wherein FIG. 4B illustrates a histogram of ejection force for CMP1 (lighter color) and MUT1 (darker color), the solid lines are Gaussian fitting, wherein FIG. 4C illustrates the probability of target ejection or probe regeneration vs template tension for the CMP1 (light) and MUT1 (dark), wherein the dotted lines are sigmoidal fitting for guidance;

FIGS. 5A-D illustrate optimization of selectivity in the SNP detection, wherein FIG. 5A illustrates histograms of ejection force for CMP4 (grey) and MUT4 (black), with the solid lines being a Gaussian fitting; FIG. 5B illustrates the probability of probe regeneration or target ejection vs template tension for CMP4 (grey) and MUT4 (black), the dotted lines being sigmoidal fitting for guidance; FIG. 5C illustrates the probability of target binding vs detection time for CMP4 (filled circles linked by dotted lines) and MUT4 (empty circles linked by solid lines, with different concentrations; FIG. 5D is a graft illustrating the time required for CMP4 (grey) and MUT4 (black) with 50% binding probability to the SNP-probe (t_{1/2}) under different target concentrations, with dotted lines being a fitting based upon the effective area of detection; and

FIG. 6 is a schematic of one embodiment of the microfluidic platform showing dimensions of the channels and the switching distance between the buffer and target channels during sensing.

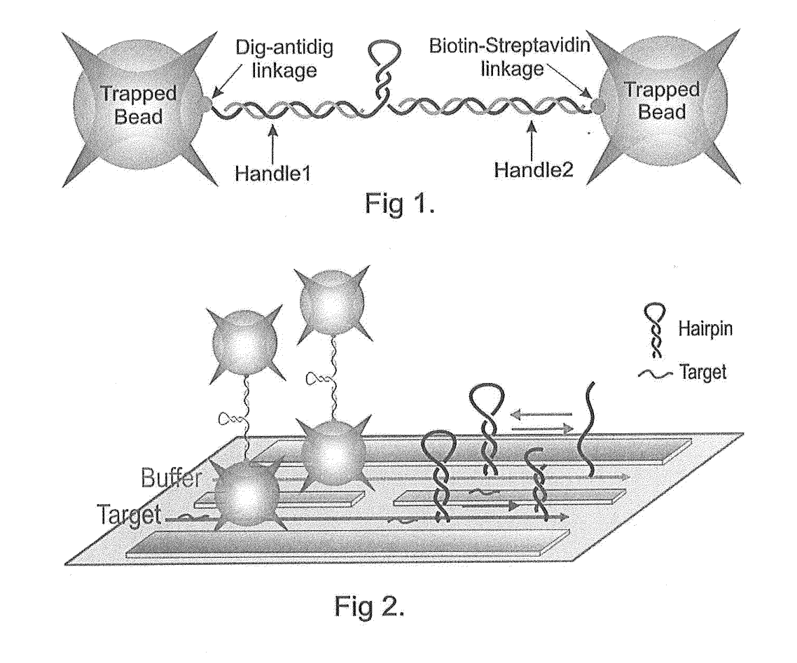

### DETAILED DESCRIPTION OF THE INVENTION

A detection device and detection methods are disclosed that utilize the stochastic behavior of a single-molecule probe to recognize and discriminate a wild type and SNP sequence in a microfluidic device using a laser tweezers instrument. The detection method utilizes on-off mechanical signals that provide little background interference and high specificity between wild type and SNP sequences. The microfluidic setting allows multiplex sensing and in-situ recycling of the SNP-probe.

As indicated herein above, the device employs a SNP-probe. The SNP-probe comprises a segment, generally referred to herein as a hairpin, that recognizes, e.g. can capture, a wild type or SNP sequence and provides a useful signal indicating recognition or capture that can be identified by the laser tweezers instrument. In a useful embodiment, the segment is a single-molecule SNP recognition sequence. The desired SNP-probe segment is located or connected between two handles, for example dsDNA handles or between two bio-polymers such as polysaccharides, polypeptides, polystyrene, among others that are each anchored to two optically trapped beads.

In a useful embodiment the handles, such as dsDNA handles of the SNP-probe are tethered to the optically trapped beads via digoxigenin (Dig)-antidigoxigenin antibody (Antidig) and biotin-streptavidin linkages, respectively.

The DNA construct for a single molecular sensing is synthesized by sandwiching a hairpin that can recognize SNP sequences between two handles, such as two dsDNA and/or biopolymer handles. Each of the handles can be labeled, for example with one or more of biotin and digoxigenin or other antigens, such as fluorescence, to be linked to specific antibodies coated on the surface of optically trapped beads. The fragment containing the SNP recognition sequence can be constructed by annealing an oligonucleotide at a suitable temperature, for example between about 90 and about 105° C., for a suitable period of time, between about 1 and about 20 minutes, followed by cooling to room temperature over a generally extended period, such as for about 0.1 to about 3 hours. The final construct can be precipitated in a solvent such as ethanol and the DNA pellet can be dissolved in water and stored, for example at a temperature of about −80° C.

A specific example of synthesis of a DNA construct is as follows. A DNA oligomer was purchased from Integrated DNA Technologies of Coralville, Iowa and purified by denaturing PAGE gel, or agraros gel, or HPLC, or chromatographic columns including capillary electrophoresis. The DNA construct for single molecular sensing was synthesized by sandwiching a hairpin that can recognize SNP sequences between two dsDNA handles. One of the DNA handles (2028 bp) was labeled with biotin at the end. This was achieved by polymerase chain reaction, PCR using a pBR322 template (New England Biolab, NEB of Ipswich, Mass. and a biotinylated primer (IDT, Coralville, Iowa), 5′-GCA TTA GGA AGC AGC CCA GTA GTA GG. The PCR product was subsequently digested with XbaI restriction enzyme (NEB). Another handle (2690 bp) was gel purified using a kit (Midsci, St. Louis, Mo.) after SacI (NEB) and EagI (NEB) digestions of a pEGFP plasmid (Clontech, Mountain View, Calif.). This handle was subsequently labeled at the 3′ end by digoxigenin (Dig) using 18 μM Dig-dUTP (Roche, Indianapolis, Ind.) and terminal transferase (Fermentas, Glen Burnie, Md.). The middle fragment containing SNP recognition sequence in the hairpin (underlined) was constructed by annealing an oligonucleotide, 5′-CTA GAC GGT GTG AAA TAC CGC ACA GAT GCG TTT GGT GCA CCG TTT TTC AGG TTT CTC TAC GGT GCA GCT TT GCC AGC AAG ACG TAG CCC AGC GCG TC with two other oligonucleotides, 5′-CGC ATC TGT GCG GTA TTT CAC ACC GT and 5′-GGC CGA CGC GCT GGG CTA CGT CTT GCT GGC at 97° C. for 5 min and slowly cooled to room temperature for 6 hours. This fragment was ligated with the 2028 bp DNA handle at one end, followed by a second ligation with the 2690 bp DNA handle using T4 DNA ligase (NEB). The final construct was ethanol precipitated and the DNA pellet was dissolved in water and stored at −80° C.

A laser tweezers instrument is utilized in the detection device for detecting the single nucleotide polymorphism. Various laser tweezers instruments are known to those of ordinary skill in the art (H. Mao, P. Luchette, Sens. Actuat. B. 129, 764-771, 2008). One example of a commercially available laser tweezers instrument is, PALM MicroTweezers IV, available from Carl Zeiss. In one useful embodiment of the invention, a diode pumped solid (DPSS) laser can be utilized as a trapping laser. More specifically, in one useful embodiment the laser has a wavelength of 1064 nm, a power of 4 W, CW mode, BL-106C and is available from Spectra-physics. P and S polarized laser light from the same laser source constituted the two laser traps. The S polarized light was controlled by a steerable mirror (Nano-MTA, Mad CityLaboratories) at a conjugate plane of the back focal plane of a focusing objective (Nikon CFI-Plan-Apochromat 60×, NA 1.2, water immersion, working distance (˜320 μm). The exiting P and S polarized beams were collected by an identical objective and detected by two position-sensitive photodetectors (PSD, DL100, and Pacific Silicon Sensor) separately. The force of the laser trap was calibrated by the Stokes force and thermal motion measurement. Both methods yielded a similar trap stiffness of ˜307 pN/(μm×100 mW) for 0.97 μm diameter polystyrene beads, available from Bangs Laboratory, Fishers, Ind.

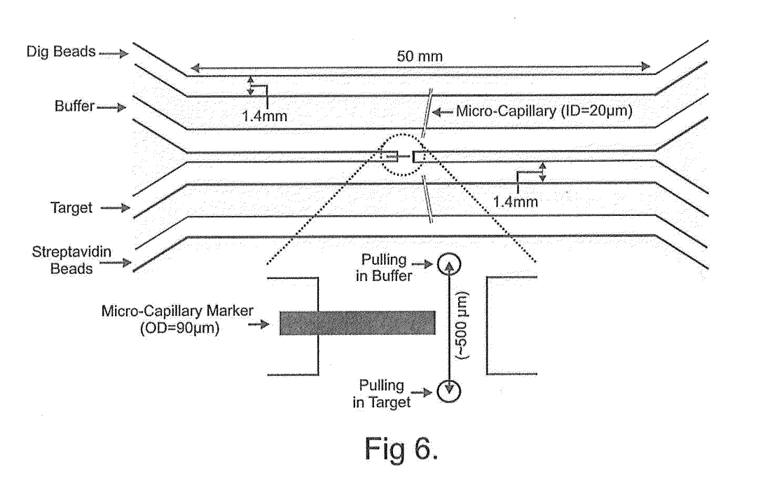

The laser tweezers instrument is operatively connected to a microfluidic device which is adapted to accept the SNP-probe. In a general form, the microfluidic device comprises different channels through which a fluid including a target DNA sequence is flowable. In a useful embodiment the microfluidic device includes at least two channels. In one useful embodiment a microfluidic device is constructed as illustrated in FIG. 6. In a useful embodiment the microfluidic device includes a buffer channel and a target channel operatively connected by a conduit to allow switching of the SNP-probe between the buffer and target channels during sensing. The conduit includes in one embodiment a micro-capillary marker having an outer diameter from about 50 to about 500 micrometers with about 90 micrometers being preferred. A conduit has a width of about 100 to about 200 micrometers in one embodiment. An additional channel including anti-digoxigenin antibody or other antibody coated beads is located adjacent to the buffer channel and connected by a conduit or micro-capillary and a further, channel, including streptavidin coated or antibody such as anti-digoxigenin antibody coated beads is located adjacent a target channel and connected by a conduit or micro-capillary. In a useful embodiment the micro-capillary has an inner diameter that ranges from about 1 to about 50 micrometers and preferably about 20 micrometers. The width of each channel can vary, and in one embodiment independently, ranges from about 0.2 to about 5 millimeters and is preferably about 1.4 millimeters. Likewise, the length of the channels can vary independently and can range from about 10 to about 300 millimeters, and in one embodiment is preferably about 50 millimeters.

Various methods can be utilized to construct a microfluidic device. For example, one method is based on soft photolithography. In one embodiment, the master features were fabricated by etching the negative photoresist (SU-8 2050, thickness 100 μm at 1600 rpm spinning rate, MicroChem Inc. Newton, Mass.) coated on a glass substrate with a SU-8 developer. This pattern was then used to prepare films of polydimethylsiloxane (PDMS) using precursor Sylgard-184 silicon elastomer base and Sylgard-184 silicon elastomer curing agent (DowCorning Corporation, Midland, Mich.) with a ratio of 10:1 under a spin rate of 1000 rpm. The PDMS film was cured at 70° C. for 2 hrs (or 55° C. overnight). This generated aPDMS film with 120 μm thickness. The injection ports for each channel were prepared by poking the film using syringe needles (Gauge 16G3/2, Becton Dickinson and Company, Franklin Lakes, N.J.). The PDMS film was then peeled off, oxygen plasma treated (1 min), and brought into contact with borosilicate coverslip (VWR) that had been treated with oxygen plasma for 1 min (plasma cleaner PDC-32G, Harrick Plasma, Ithaca, N.Y.).

In a second method, the microfluidic chamber is prepared by sandwiching a patterned Nesco-film (Azwell, Osaka, Japan) between two glass coverslips (VWR). The microfluidic patterns (FIG. S1) were designed in CorelDraw (Corel Corporation) and imprinted into the Nesco-film directly by a laser cutter (VL-200, Universal Laser Systems, Scottsdale, Ariz.). The patterned Nesco-film and the two coverslips were thermally sealed at 155° C. The thickness of the film thus treated (100±5 μm) determined the channel thickness. Samples were injected into microfluidic channels through the holes in one of the coverslips prepared by the same laser cutter. To transport the beads attached with DNA samples and the streptavidin coated beads into buffer or target channels, microcapillary tubes (ID 20 μm, OD 90 μm) were used. The same tube was used as a separation marker in the conduit between the target and buffer channels. The distance for SNP-probe to switch between the buffer and target channels through the conduit can range from about 50 to about 2000 μm, and is about 500 μm in a useful embodiment.

Characterization of the hairpin of the SNP-probe can be accomplished in one embodiment as follows. Anti-Dig antibody-coated polystyrene beads (diameter: 2.17 μm, Spherotech, Lake Forest, Ill.) were incubated with diluted DNA construct obtained above (˜1 ng/μL) in 100 mM NaCl, 10 mM tris buffer pH 7.4 for 1 h at 23° C. to attach the DNA construct via the Dig/anti-Dig complex. Beads coated with streptavidin (diameter; 0.97 μm, Bangs Laboratory) were dispersed into the same buffer and injected into the reaction chamber. These two types of beads were trapped separately using two laser beams. To immobilize the DNA construct between the two beads, the bead already attached with the DNA construct was brought close to the bead coated with streptavidin by the steerable mirror in the laser tweezers instrument. Once the DNA tether was trapped between the two beads, the Nano-MTA steerable mirror that controls the anti-Dig-coated bead was moved away from the streptavidin-coated bead, in one embodiment with a load speed of ˜5.5 pN/s. The hairpin structure with the SNP recognition sequence was unfolded when tension inside the tether was gradually increased. Unfolding events with sudden change in the end-to-end distance were observed during the process. Single tether was confirmed by a single breakage event when the DNA was overstretched. The rupture force was measured directly from the force vs. extension curves while the change in contour length (ΔL) due to the unfolding was calculated by the two data points flanking a rupture event using an, extensible worm-like chain (WLC) model (Equation1).

$$\frac{x}{L} = 1 - \frac{1}{2} {( \frac{{k}_{B} T}{FP} )}^{\frac{1}{2}} + \frac{F}{S} \qquad ( 1 )$$

where x is the end-to-end distance, k_{B} is the Boltzmann constant, T is absolute temperature, P is the persistent length (51.95 nm), F is force, and S is the elastic stretch modulus (1226 pN). When the molecule was relaxed with the same loading speed, the hairpin was refolded in the lower force region (<10 pN). The refolding was manifested by a sudden change in force or end-to-end distance in the force vs. extension curve. The stochastic bistate transition (or hopping) of the hairpin was observed with a fixed distance between the two laser traps. By adjusting this distance, the hairpin containing sequence can populate either in the folded or unfolded states. In the SNP sensing, the distance can be adjusted to populate the hairpin in an unfolded state to facilitate the binding of the SNP target.

The unfolding force of the hairpin containing the example target DNA recognition sequence was 9.5±0.1 pN. Taking into account of the GC content and the length of the hairpin stem, the observed unfolding force matches well with the results observed previously, see M. T. Woodside et al. Proc. Natl. Acad. Sci. U.S.A. 103, 6190-6195, 2006. The change in contour length (ΔL) as a result of hairpin unfolding was 13.4±0.1 nm. The contour length per nucleotide was calculated according to the following equation.

ΔL=N×Lₙₜ−Δx (2)

Where N is the number of nucleotides contained in the structure (35 nt), Lₙₜ is the contour length per nucleotide, and Δx is the end-to-end distance (2 nm, the diameter of dsDNA). According to this calculation, the value for Lₙₜ was found to be 0.44±0.01 nm, which is in good agreement with the previous studies, see M. T. J. Record, C. F. Anderson, T. M. Lohman, Quart. Rev. Biophys. 11, 103-178, 1978; J. B. Mills, E. Vacano, P. J. Hagerman, J. Mol. Biol. 285, 245-257 1999; M. T. Woodside et al. Proc. Natl. Acad. Sci. U.S.A. 103, 6190-6195, 2006).

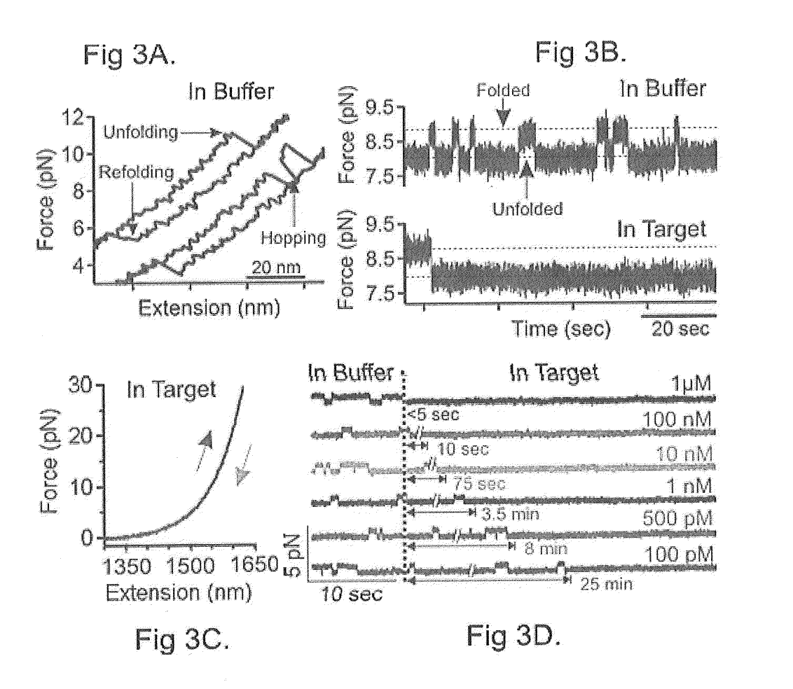

Once the SNP-probe has been constructed, the same can be connected to the microfluidic device in order to detect SNP targets. In a useful embodiment, the SNP-probe is placed inside a microfluidic device having interconnected channels, such as shown in FIG. 2 and FIG. 6. Utilizing a microfluidic device having interconnected channels allows desired buffers to be utilized in separate channels while keeping free movement of the SNP-probe between channels. Before sensing, the tethered DNA construct is stretched and relaxed repeatedly, such as from about 1 to about 10 times, which allows unfolding and refolding of the hairpin of the SNP-probe. Hopping between the folded or “on” state and the unfolded or “off” state of the hairpin is observed by the laser tweezers instrument at fixed positions of the two laser traps, see FIG. 3b for example. The distance between the two laser traps can be adjusted to allow the bi-state stochastic hopping of the hairpin in the buffer channel. The on/off behavior can be exploited for subsequent detection of a desired target when a target sample including a wild type or SNP sequence is introduced into a channel of the microfluidic device, the hairpin is populated in its unfolded state and the hopping ceases. Generally, the greater the concentration of the wild type or SNP sequence in the target sample, the shorter the time period needed to detect the same. As both wild type and SNP sequences are able to bind to the hairpin of the SNP-probe, it is desirable to distinguish between such bindings.

In one method, to distinguish the binding of a wild type sequence and a SNP sequence, a force is applied to the hairpin bound with either of the sequences in a channel of the microfluidic device. In some embodiments, a small rupture event is observed above a rupture force. It is believed that the rupture event represents the ejection of the bound target probably due to the force induced melting. It can be confirmed that the bound target has been ejected by relaxing the probe to a lower force region, and hairpin refolding can be observed after ejection. The ejection of the bound target forebodes the regeneration of the SNP-probe at the lower force range. It has been found that the ejection force for a SNP sequence is less than the force needed to eject a wild type sequence. Once the SNP-probe is free from a bound target, it can be used for a next round of detection, unless a tether of the probe is broken.

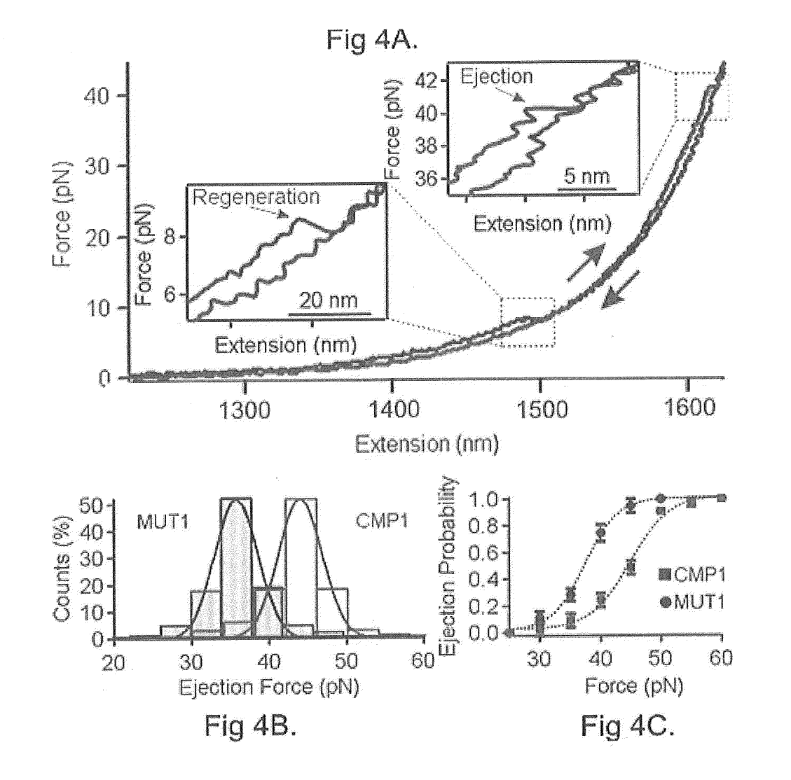

One suitable method for target ejection is as follows. Once the SNP-probe was bound with a DNA target in the target channel, the complex is moved to the buffer channel, see FIG. 2 and stretched to a fixed force, e.g. for about 30 to about 60 pN, to allow the ejection of the bound target. The ejection is manifested by a sudden decrease in the force. It can be confirmed when the tether is relaxed to the lower force region (<10 pN) where the hairpin refolding was observed, see FIG. 4A. Once the SNP-probe was free from the bound target, it can be used for a next round of detection until the tether is broken. The target ejection probability or probe regeneration probability can be calculated based on the number of the curves with the target ejection event vs. the total curves with the same maximal stretching force in a force titration experiment. It can also be calculated by the total number of curves below specific force vs. overall curves integrated from a histogram of the ejection force, see FIG. 4B. These two methods showed identical results in experimental tests.

Two methods are described herein that can be utilized to determine the target detection time. In the first method, the trap to trap distance is adjusted to allow the hopping of the hairpin of the SNP-probe in the buffer channel. Time zero is defined as the moment the SNP-probe and the two trapped beads are moved together to the target channel in which a target DNA (either wild type or SNP sequence) with a specific concentration is flowed. The binding of the target DNA at specific time (detection time) was revealed by the cease of the hopping to the unfolded state.

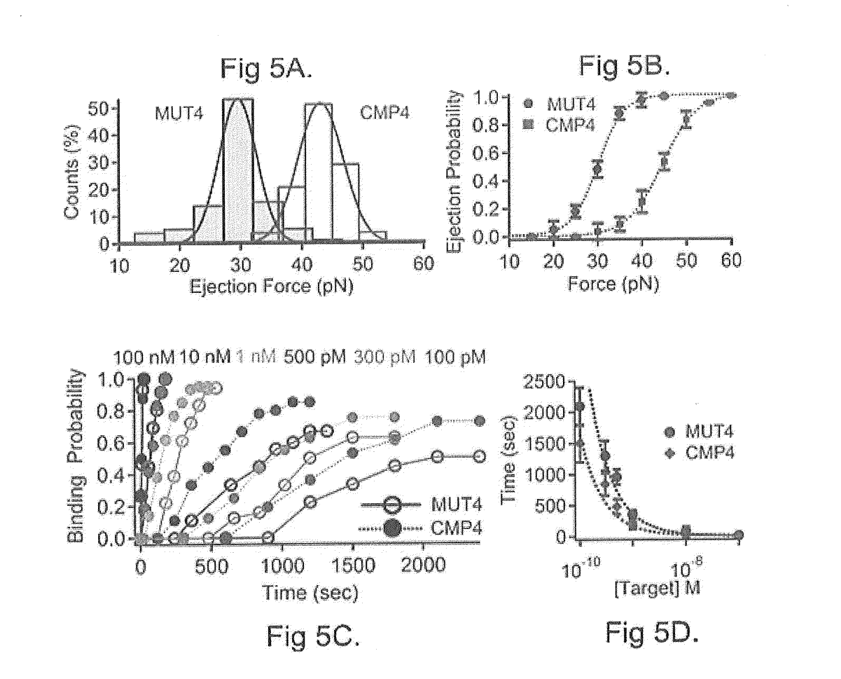

In the second method, see FIG. 5C, the hairpin in the SNP-probe is populated in the unfolded state by adjusting the trap-to-trap distance in the buffer channel. Time zero is defined as the moment the SNP-probe and the two trapped beads are moved together to the target channel in which a target DNA (either wild type or SNP sequence) with a specific concentration is flowed. At a specific time interval, the SNP-probe is stretched to 20 pN and relaxed towards 0 pN. The absence of hairpin refolding events below 10 pN indicates the binding of the target. DNA. Once binding is recorded, the probe is moved to the buffer channel and stretched to higher force (˜50 pN) for target ejection and regeneration of the SNP-probe as described above. If the binding is not observed, the SNP-probe is subjected to another time interval in the target channel for DNA binding. Binding probability at a particular time, see FIG. 5C is calculated as the percentage of the SNP-probes with binding events vs overall SNP-probes surveyed in that time period.

Although both methods showed similar results during experimentation, the second approach was more reliable as trap-to-trap distance was not required to remain constant, which is a demanding task for long term experiments.

Assuming that target binding to the probe is a diffusion controlled process within an effective detection area of A_{effective}, the detection time for the probe to recognize a target is determined by the time interval between the two target molecules that subsequently flow through this area. The number of target molecules that flow through this area per minute is given by,

$$\begin{matrix}\frac{{v}_{flow} \times C \times {N}_{A} \times {A}_{effective}}{{A}_{total}} = \frac{\begin{matrix}5 \times {10}^{- 7} liter \text{/} min \times 6.02 \times \\ {10}^{23} moleule \text{/} mole \times C mole \text{/} liter \times \\ {A}_{effective} {m}^{2}\end{matrix}}{1.7 \times {10}^{- 7} {m}^{2}} \\ = 1.8 \times {10}^{24} \times C \times {A}_{effective} molecule \text{/} min\end{matrix} \qquad ( 3 )$$

Where v_{flow} is the flow rate of the buffer in each channel, which was maintained at 5×10⁻⁷ liters/min by a Harvard 2000 pump (Harvard Apparatus, Holliston, Mass.), C is the target concentration, NA is the Avogadro's number, and A total, 1.7×10⁻⁷ m² is the cross section of the channel. Consequently, the time required for single target molecule to pass through the effective-detection area with 50% probability (t_{1/2}, or detection half time) can be calculated as,

$${t}_{1 / 2} = \frac{1 \times 50 \%}{1.8 \times {10}^{24} \times C \times {A}_{effective}} = \frac{2.8 \times {10}^{- 25}}{C \times {A}_{effective}} min \qquad ( 4 )$$

Equation 4 was used to fit in the curves shown in FIG. 5D. From the fitting, the value of the effective detection area was found to be 70 nm² and 41 nm² for CMP4 and MUT4, respectively.

Although selectivity can be estimated by the ratio of the ejection probability between CMP and MUT, see FIGS. 4C and 5B, this method is not exact, as the ejection probability evolves with stretching force. Here Boltzmann distribution can be used to estimate the selectivity of CMP over MUT at 50% ejection probability. The ratio of the SNP-probe bound with MUT, P_{MUT}, to the SNP-probe bound with CMP, P_{CMP}, is given by,

$$\begin{matrix}\frac{{P}_{MUT}}{{P}_{CMP}} = exp - ( \frac{{E}_{CMP} - {E}_{MUT}}{{k}_{B} T} ) \\ = exp - ( \frac{Δ {G}_{CMP , ejection} - Δ {G}_{MUT , ejection}}{{k}_{B} T} )\end{matrix} \qquad ( 5 )$$

Where kB is the Boltzmann constant, T is absolute temperature, E_{CMP} and E_{MUT} are energies of SNP-probe bound with CMP and MUT; respectively. The energy difference is approximated by the difference between the change in the free energy for ejection of CMP, Δ_{CMP, ejection}, and that for MUT, ΔG_{MUT, ejection}, which, in turn, can be calculated through Jarzynski's theorem for non-equilibrium systems. This calculation provided the selectivity ratio of 80 to 1 for CMP1 over MUT1 and 1600 to 1 for CMP4 over MUT4.

### EXAMPLES

In order to illustrate the devices and methods of the present invention, the single nucleotide polymorphism SNP R_{S}133049 was selected for sensing. The indicated SNP has been associated with coronary heart diseases. A tethered SNP-probe containing the indicated sequence, bound as described hereinabove, was placed inside a microfluidic device with interconnected channels having the structure shown in FIGS. 2 and 6. The device design allows desired buffers in separate channels while keeping free movement of the SNP-probe between channels. Before sensing, the tethered DNA construct was repeatedly stretched and relaxed, which allowed unfolding and refolding of the hairpin in the SNP-probe, respectively, see FIG. 3A. Hopping between folded, or “on”, and unfolded, or “off”, states of the hairpin was also observed at fixed positions of the two laser traps, see FIG. 3B. Analysis of the change in contour length and rupture force confirmed the hairpin structure in the DNA construct.

In our first design of a SNP-probe, each end of the 19-nt probe extended 2-nt into the hairpin stem. The distance between the two laser traps was adjusted to allow the bi-state stochastic hopping of the hairpin in the buffer channel, see FIG. 3B, top panel. This on-off behavior was exploited for subsequent detection of oligodeoxynucleotide (ODN) targets. When the SNP-probe was moved to the channel that contained a complementary 19-nt ODN, CMP1, 5′-GTA GAG AAA CCT GAA AAA C, with 1 μM concentration, hopping immediately ceased and the hairpin populated in its unfolded state, see FIG. 3B, bottom panel. In contrast; hopping of the hairpin persisted for up to 35 min in the presence of a non-complementary ODN, NCMP, 5′-TTT TCA GGT TTC TCT. These observations were consistent with the specific binding of the CMP1 to the probe, which eliminated the hopping. The specific binding was further supported by the absence of unfolding and refolding features in the force vs. extension curves in the presence of CMP1; see FIG. 3, while these features were not affected in 1 μM NCMP solutions.

To facilitate the binding of CMP1, we varied the concentration of CMP1 under the trap-to-trap distance that favored an unfolded hairpin. As shown in FIG. 3D, the time required to catch a CMP1 molecule (indicated by two-headed arrows) was inversely dependent on the CMP1 concentration. Given enough time, it is expected to detect infinitely low concentration of the CMP1. However, due to the limit of the effective detection area vs cross-section of the microfluidic channel (˜50 nm² vs 100,000 μm², see below), we were able to detect 100 pM targets in 30 min. Surprisingly, when an SNP sequence MUT1, 5′-GTA GAG AAA CGT GAA AAA C, was tested, similar binding behavior and detection limit were observed.

To distinguish the binding of MUT1 from CMP1, we applied a force up to 60 pN on the hairpin bound with either of the two targets in the buffer channel. During this process, we observed a small rupture event (See FIG. 4A) at the force above 25 pN. When the tension was relaxed, the refolding of the hairpin was almost always observed at the lower force region (<10 pN, FIG. 4A). We surmise the rupture event represents the ejection of the bound target probably due to the force induced melting. The observed change in contour length (ΔL) of the ejection events matches well with the value calculated when bound DNA target is lost. The ejection of bound targets, therefore, forebodes the regeneration of the SNP-probe at the lower force range. When we compared the ejection forces for CMP1 and MUT1, we found the former required significantly higher value than the latter (44.0±0.8 pN vs 35.5±0.5 pN, FIG. 3B). “Force titration” experiments in which maximal extending forces were increased 5 pN each time were performed to estimate the probability of ejection (or regeneration) in each force range. The result (FIG. 4C) was identical with that obtained by the integration of the histograms in FIG. 4B. Based on the 50% ejection probability, we calculated the selectivity between CMP1 and MUT1 as 80:1.

To increase the specificity, we selected shorter DNA targets with the expectation that single site mutation will be more pronounced. However, binding was not observed for 10-nt targets CMP2, 5′-GAA ACC TGA A & MUT2, 5′-GAA ACG-TGA A at the concentration as high as 10 μM. For 15-nt sequences CMP3, 5′-AGA GAA ACC TGA AAA & MUT3, 5′-AGA GAA ACG TGA AAA, target binding that prevents the hopping of the hairpin in the SNP-probe only occurred at the concentration above 100 nM.

To increase the strength of target binding, we selected 15-nt sequences CMP4, 5′-CCT GAA AAA CGG TGC & MUT4, 5′-CCT GAA CTG TGC, that recognize both the stem and the loop of the hairpin probe. This strategy demonstrated dramatic improvement in the detection limit and the selectivity. The ejection force analysis showed that the probe bound with CMP4 required 42.5±1.2 pN Whereas that with MUT4 required 29.5±1.5 pN to eject the target (FIG. 5A). The difference between these two ejection forces (13.0±1.9 pN) is significantly higher than that for the 19-nt targets (8.5±0.9 pN). The analysis on the regeneration probability also demonstrated increased difference between these two targets at specific force (FIG. 5B). Subsequent calculation revealed a remarkably increased selectivity of 1600:1 between CMP4 and MUT4.

Next, we measured the time required for the SNP-probe to catch either CMP4 or MUT4. To this purpose, we adjusted the trap-to-trap distance to populate the hairpin in its unfolded state in the buffer channel. We then exposed the SNP-probe to CMP4 or MUT4 in separate microfluidic channels with 100 nM-100 pM target concentrations. The binding of a specific target was revealed by the absence of hairpin refolding event in the force vs. extension curves collected at certain time interval. The probe was regenerated at higher forces for the next round of detection. FIG. 5C depicts that for concentrations below 10 nM, the binding for CMP4 takes less time compared to MUT4, indicating it is easier for the SNP-probe to recognize a complementary sequence than a mutant. Assuming a diffusion-controlled target recognition process in an effective detection area of A_{effective}, we calculated the time for 50% probability of target binding (or half time, t1/2) based on the irate of the target molecules that enter this area,

$${t}_{1 / 2} = \frac{{A}_{total}}{2 \times {v}_{flow} \times C \times {N}_{A} \times {A}_{effective}} \qquad ( 6 )$$

Where v_{flow} is the flow rate of the buffer in a microfluidic channel, C is the target concentration, N_{A} is the Avogadro's number, and Aₜₒₜₐₗ is the cross section of the channel. This expression gave good fitting to the curves shown in FIG. 5D, which reveals that the detection half time increases with decreasing target concentration. The fitting yielded A_{effective} of 70 nm² for CMP4 and 41 nm² for MUT4 recognition. This result confirmed that CMP4 can be recognized more efficiently than MUT4. The A_{effective} values are within the range expected for the SNP hairpin, which validates our model. Eqn 6 also implies that with increased flow rate and decreased size of a microfluidic channel, t_{1/2} can be effectively reduced to detect targets with even lower concentrations.

In summary, we have successfully demonstrated a novel single molecule SNP detection method using stochastic mechanical signals. The noise free mechanical signal warrants superior sensitivity for this approach. The on-off state of the detector can be adjusted by the control of the tension in the SNP-probe, which also effectuates the in situ recycling of the sensor. As a proof of concept, we were able to detect, 100 pM of an SNP target in 30 minutes. Given enough time, this method has the potential to detect much lower target concentration inside smaller microfluidic channels. The microfluidic platform allows multiplex sensing after the incorporation of additional channels. In fact, we have successfully tested this, capability in a 5-channel microfluidic device. This technique is not only applicable to detect SNP, but also amenable to serve as a generic on-off digital biosensor, by using specific recognition elements such as DNA aptamers for example.

While in accordance with the patent statutes the best mode and preferred embodiment have been set forth, the scope of the invention is not intended to be limited thereto, but only by the scope of the attached claims.

What is claimed is:

1. A device for detecting a single nucleotide polymorphism (SNP), comprising:
A SNP-probe including a hairpin that recognizes a target DNA comprising one or more of a wild type and SNP sequence;
A microfluidic device; and
A laser tweezers device operatively connected to the microfluidic device for force base stochastic sensing of the one or more of the wild type and the SNP sequence.

2. The device according to claim 1, wherein the microfluidic device has at least two channels including a buffer channel and a target channel through which fluids are flowable.

3. The device according to claim 2, wherein the buffer channel and target channel are operatively connected by a conduit to allow switching of the SNP-probe between the buffer channel and target channel.

4. The device according to claim 2, wherein the SNP-probe including the hairpin is placed inside the microfluidic device and the hairpin can be folded and unfolded in one of the channels.

5. The device according to claim 4, wherein a single-molecule of the hairpin comprises a SNP recognition sequence.

6. The device according to claim 5, wherein the hairpin is located between two dsDNA handles or between two bio-polymers that are each anchored to two optically trapped beads.

7. The device according to claim 6, wherein the dsDNA handles of the SNP-probe are tethered to the optically trapped beads via digoxigenin-antidigoxigenin antibody and biotin-streptavidin linkages, and wherein the laser tweezers comprises a diode pumped solid laser.

8. The device according to claim 3, wherein each channel has a width that independently ranges from about 0.2 to about 5 millimeters, and wherein the length of each channel independently ranges from about 10 to about 300 millimeters, wherein the conduit that connects two channels has a width of about 100 to about 200 micrometers.

9. A method for detecting a single nucleotide polymorphism (SNP), comprising the steps of:
Obtaining a SNP detection device including a microfluidic device operatively connected to a laser tweezers device;
Operatively connecting a SNP-probe containing a hairpin that recognizes a SNP sequence to the SNP detection device; and
Measuring a force exerted by the SNP-probe in the SNP detection device in the presence of a target sample and determining whether the SNP sequence is present in the target sample.

10. The method according to claim 9, wherein said measuring the force comprises measuring an unfolding force of the hairpin which comprises a target DNA recognition sequence.

11. The method according to claim 9, wherein the microfluidic device has at least two channels including a buffer channel and a target channel through which fluids are flowable, and further including the step of moving the SNP-probe between the buffer channel and the target channel.

12. The method according to claim 11, further including the step of introducing a target sample in a fluid into the target channel of the microfluidic device and further folding and unfolding the hairpin in the target channel.

13. The method according to claim 11, further including the step of allowing binding of a wild type or SNP sequence to the hairpin, and further ejecting the wild type or SNP sequence from the hairpin by stretching the hairpin bound with the wild type or SNP sequence.

14. The method according to claim 13, further including the step of measuring the ejection force.

15. The method according to claim 13, further including the step of reusing the SNP-prove after ejection of the wild type or SNP sequence.

16. A method for detecting a single nucleotide polymorphism (SNP), comprising the steps of:
Obtaining a SNP detection device including a microfluidic device operatively connected to a laser tweezers device;
Connecting a SNP-probe containing a hairpin that recognizes a wild type or SNP sequence to the SNP detection device; and
Flowing a target sample through a channel of the microfluidic device; and
Folding and unfolding the hairpin in said channel.

17. The method according to claim 16, further including the step of determining whether the SNP sequence is present in the target sample.

18. The method according to claim 17, wherein the microfluidic device has at least two channels including a buffer channel and a target channel through which fluids are flowable, and further including the step of moving the SNP-probe between the buffer channel and the target channel.

19. The method according to claim 18, further including the step of measuring an unfolding force exerted by the SNP-probe.

20. The method according to claim 16, further including the step of binding a wild type or SNP sequence to the hairpin, and further ejecting the wild type or SNP sequence from the hairpin by stretching the hairpin-wild type or hairpin-SNP sequence.
